(19) United States
(12) Patent Application Publication
(10) Pub. No.: US 2008/0003194 A1
(43) Pub. Date: Jan. 3, 2008

(54) USE OF GUM ARABIC AND/OR MODIFIED GUM ARABIC SOLUTIONS TO REMOVE ODOR CAUSING MOLECULES

(75) Inventor: Louis Rebrovic, Racine, WI (US)

Correspondence Address: S.C. JOHNSON & SON, INC., 1525 HOWE STREET, RACINE, WI, 53403-2236, US

(73) Assignee: S.C. JOHNSON & SON, INC, Racine, WI (US)

(21) Appl. No.: 11/765,982

(22) Filed: Jun. 20, 2007

Related U.S. Application Data
(60) Provisional application No. 60/816,949, filed on Jun. 28, 2006.

(51) Int. Cl. A61L 9/01; A61L 9/013

(52) U.S. Cl. 424/76.1

(57) ABSTRACT

An aqueous deodorizing composition is disclosed for removing malodors from the air as well as soft surfaces such as carpeting, upholstery, bedding, draperies, clothing and other fabrics. The aqueous solution includes gum arabic and/or modified gum arabic, such as by ion-exchange or chemical modification. The composition may also include an oxidant such as iodine to improve the malodor removal performance thereof. Optionally, the composition may further comprise one or more surfactants such as sodium lauryl sulfate.

### CROSS-REFERENCE TO RELATED APPLICATION

This application is a non-provisional U.S. application which is based on and claims priority from provisional Application Ser. No. 60/816,949, filed on Jun. 28, 2006.

### BACKGROUND

1. Technical Field

Solutions comprising water and gum arabic and/or modified gum arabic are disclosed for use in removing and/or treating malodors.

2. Description of the Related Art

Gum arabic is a natural gum prepared from an exudate derived from the stems and branches of sub-Saharan Acacia senegal and Acacia seyal trees. It is produced naturally as large nodules during a process called gummosis to seal wounds in the balk of the tree. Gum arabic is a less consistent material than other hydrocolloids. Gum arabic is generally used as an emulsifier for formulating various hydrocolloidal compositions, as well as a stabilizer in food and drink industry. Use of gum arabic as a primary active ingredient in a deodorizing composition has yet to be developed.

Nevertheless, a wide variety of deodorizing compositions are known in the art to remove malodors existing in the air and/or on fabric surfaces. In general, airborne and fabric malodors are most commonly caused by environmental odors such as tobacco odor, cooking and/or food odors, or body odor. The unpleasant odors are mainly organic molecules which have different structures and functional groups, such as amines, acids, alcohols, aldehydes, ketones, phenols, polycyclics, indoles, aromatics, polyaromatics, etc. Fabric malodorants can also be made up of sulfur-containing compounds, such as, mercaptans, sulfides and/or disulfides.

One type of deodorizing composition uses per fumes to mask malodor. Odor masking is the intentional concealment of one odor by the addition of another. The masking of odors is typically accomplished by using perfumes or fragrances. However, high levels of fragrance are needed to ensure that the malodor is no longer noticeable, or suitably masked and the masking techniques do nothing to remove or modify the source of the odor.

Odor modification, in which the odor is changed, e.g., by chemical modification, has also been used. Malodor modification methods known in the art include oxidative degradation and reductive degradation. Oxidative degradation employs oxidizing agents such as oxygen bleaches, chlorine, chlorinated materials such as sodium hypochlorite or chlorine dioxide, and potassium permanganate to reduce malodors. Reductive degradation, on the other hand, employs reducing agents such as sodium bisulfite to reduce malodors. Both oxidation and reduction strategies are unacceptable for general use on fabric because they can damage colored fabrics.

Other modification techniques involve antibacterial and antifungal agents which regulate the malodor-producing microorganisms found on the surface to which the deodorizing composition is directed. Many skin deodorant products use this technology. These compositions are not effective on malodors that have already been produced and malodors that do not come from bacterial sources, such as tobacco or food odors.

Other methods of odor control utilize actives that react with malodors having specific functional groups Examples of such actives include biguanide polymers, which complex with organic compounds containing organically bound N and/or S atoms and fatty alcohol esters of methyl methacrylic acid which react with thiols, amines, and aldehydes. Such actives are limited in the scope of protection which they afford because they only react with limited types of malodor.

Film forming polymers have been used to blanket or entrap malodors and keep the malodor molecules from vaporizing, thereby reducing the partial pressure of the malodorant and reducing its perceptibility to the human sense of smell. However, film forming polymers are not preferred for many applications because they leave a visible residue when used on upholstery, carpeting or other fabrics.

In contrast to a masking or chemical reaction material, some odor absorbing materials eliminate a broad spectrum of malodorous molecules. Known solid odor absorbers such as activated charcoal and zeolites can be harmful to fabrics and therefore are not preferred as an odor controlling agent under these circumstances. Furthermore, zeolites can impart a “harsh” feel if too much zeolite is deposited on a surface.

Cyclodextrin molecules have been used to treat fabric odors. Cyclodextrins comprise varying numbers of glucose units that form a cage structure. Cyclodextrins absorb malodors by trapping or caging odor-causing molecules in their cage structure. However, some small molecules are not sufficiently absorbed by the cyclodextrins because the cavity of the cyclodextrin molecule may be too large to adequately hold or contain a smaller organic molecule. Moreover, cyclodextrins can be a breeding ground for certain microorganisms, especially when applied as aqueous compositions. Finally, cyclodextrins are generally ineffective in treating airborne odors.

Certain glycols, such as triethylene glycol (TEG), have also been used for sanitizing air and eliminating airborne odors caused by bacterial as TEG is capable of killing airborne bacteria.

Despite all of the above efforts at odor control, there is still a need for improved methods for controlling odors both in air and on surfaces (i.e., carpeting, upholstery, drapes, clothing, bedding, etc.), which are safe and effective for use where the active ingredients may come into contact with a consumer's body. Further, a deodorizing composition that effectively works on a broad range of malodorants both in air and on surfaces without the above-mentioned drawbacks is needed.

### SUMMARY OF THE DISCLOSURE

In satisfaction of the above noted needs, a deodorizing composition comprising a malodor counteractant, such as gum arabic, for removing, eliminating or counteracting airborne and/or surface malodors is disclosed.

Preferably, the disclosed composition comprises water and gum arabic and is suitable for distributing into the air or onto the surface where the malodors exist.

The gum arabic is preferably present in the disclosed composition at a concentration of from about 0.1 to about 50 wt %, more preferably from about 1 to about 10 wt %, and most preferably from about 1 to about 5 wt %.

The disclosed composition may further comprise a co-solvent such as an alcohol. The content of the alcohol preferably ranges from about 1 to about 20 wt %. In a refinement, the alcohol is ethanol and is present in an amount of from about 5 to about 10% by weight of the composition.

In an improvement, the disclosed composition further includes an additional malodor counteractant, such as iodine, for further improving the odor removal performance thereof. In a refinement, iodine is present in the composition at a concentration of from about 0.001 to about 1 wt %.

Preferably, the additional malodor counteractant has a synergic effect with the gum arabic. This effect, however, may be dependent on the specific malodorant that is being treated.

In a refinement of this concept, gum arabic and iodine are dissolved in a solvent mixture of an alcohol and water, wherein the alcohol improves the solubility of iodine.

In another improvement, the disclosed composition further optionally includes one or more surfactants. A preferred surfactant is sodium dodecyl sulfate. Alternative surfactants apparent to those skilled in the art can also be used. In a refinement of this concept, sodium dodecyl sulfate (a.k.a. sodium lauryl sulfate) is included in the composition and is present in an amount of about 1 wt %.

In a preferred embodiment, the disclosed composition comprises about 3 wt % gum arabic, about 0.03 wt % iodine, about 1 wt % sodium lauryl sulfate, and the balance being water.

As a further improvement of the disclosed composition, the gum arabic may be modified to improve its odor removal performance.

In a refinement, the disclosed composition may comprise gum arabic modified by replacing hard water cations contained therein, such as calcium and magnesium, with softer cations such as sodium and/or potassium.

In another refinement, the disclosed composition comprises gum arabic modified by converting pendent hydroxyl groups of the gum arabic to functional groups that react with the malodorant(s).

Methods of modifying gum arabic by ion exchange or chemical transformation are also disclosed.

Other advantages and features of the disclosed methods and compositions will be described in greater detail below.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 graphically illustrates the ability of solid gum arabic to reduce the ground level concentration (GLC) of iso-butylamine and its comparison with reduction achieved by solid cyclodextrins (a-CD, b-CD and g-CD);

FIG. 2 graphically illustrates the ability of solid gum arabic to reduce the ground level concentration (GLC) of pyridine and its comparison with reduction achieved by solid cyclodextrins (a-CD, b-CD and g-CD);

FIG. 3 graphically illustrates the ability of gum arabic solution to reduce the GLC of ethylpyrazine and its comparison with b-cyclodextrin;

FIG. 4 graphically illustrates the dependence of GLC reduction of ethylpyrazine on concentration of gum arabic in a mixture of 10% ethanol and 90% water;

FIG. 5 graphically illustrates the dependence of GLC reduction of iso-butylamine on concentration of gum arabic in a mixture of 10% ethanol and 90% water;

FIG. 6 graphically illustrates the dependence of GLC reduction of pyridine on concentration of gum arabic in a mixture of 10% ethanol and 90% water;

FIG. 7 graphically illustrates the ability of gum arabic solution in water to reduce the GLC of 3-ydroxy-1-octene and to convert the 3-ydroxy-1-octene to 3-octanone, and its comparison with b-cyclodextrin;

FIG. 8 graphically illustrates the synergetic effect of combining iodine with gum arabic to reduce the GLC of nonyl aldehyde;

FIG. 9 graphically illustrates the ability of gum arabic solution to reduce the GLC of ethylpyrazine and its comparison with b-cyclodextrin;

FIG. 10 graphically illustrates the comparison between the odor removal performance of a preferred composition according to this disclosure and that of a commercial composition containing beta-cyclodextrin against six common malodorants;

FIG. 11 graphically illustrates the ability of a preferred composition according to this disclosure to reduce the perception of common malodors by human sense of smell;

FIG. 12 graphically illustrates the comparison between the odor removal performance of a preferred composition according to this disclosure with that of other existing deodorizing compositions against smoke malodor;

FIG. 13 graphically illustrates the comparison between the odor removal performance of a preferred composition according to this disclosure with that of other existing deodorizing compositions against low level mold and mildew odor;

FIG. 14 graphically illustrates the comparison between the odor removal performance of a preferred composition according to this disclosure with that of other existing deodorizing compositions against high level mold and mildew odor;

FIG. 15 graphically illustrates the comparison between the odor removal performance of a preferred composition according to this disclosure with that of other existing deodorizing compositions against low level cat urine odor;

FIG. 16 graphically illustrates the comparison between the odor removal performance of a preferred composition according to this disclosure with that of other existing deodorizing compositions against high level cat urine odor;

### DETAILED DESCRIPTION OF THE PRESENTLY PREFERRED EMBODIMENTS

In a general embodiment, the disclosed composition comprises a malodor counteractant, such as gum arabic, for removing airborne and surface malodors. For general purpose of this disclosure, the term “odor removal” refers to the overall reduction of the odor perceived by human sense of smell, and is interchangeable with “odor suppressing”, “odor elimination”, “odor counteracting”, or the like.

Gum arabic suitable for use in the disclosed composition is a complex and variable mixture of polysaccharides and glycoproteins. Depending on the source (Acacia seyal or Acacia senegal), the polysaccharide components contain a greater proportion of L-arabinose relative to D-galactose (Acacia seyal) or D-galactose relative to L-arabinose (Acacia senegal). The gum from Acacia seyal also contains significantly more 4-O-methyl-D-glucuronic acid but less L-rhamnose and unsubstituted D-glucuronic acid than that from Acacia senegal.

In general, gum arabic consists of a mixture of lower molecular weight polysaccharide (MW ˜0.25×10⁶; major component) and higher molecular weight hydroxyproline-rich glycoprotein (MW ˜2.5×10⁶ minor component). Depending on the source, gum arabic may also contain peroxidases and/or oxidases, as well as metal cations such as magnesium, calcium, and potassium ions. Because it is a mixture of polymers at various concentrations, gum arabic varies significantly with source, the exact molecular structures are uncertain.

The glycoprotein of gum arabic is a high molecular weight hydroxyproline-rich arabinogalactan (˜2% protein) containing a repetitive and almost symmetrical 19-residue consensus motif-ser-hypa-hypa-hypa-thr-leu-ser-hypb-ser-hypb-thr-hyp-thr-hypa-hypa-hypa-gly-pro-his- with contiguous hydroxyprolines attached to oligo-a-1,3-L-arabinofurans and non-contiguous hydroxyprolines attached to galactose residues of oligo-arabinogalactans, in combination with a b-1,3-galactopyran core and rhamnoglucuronoarabinogalactose pentasaccharide side chains.

Gum arabic is generally used as a hydrocolloid emulsifier, texturizer and film-former. The presence of hydrophilic carbohydrate and hydrophobic protein provides the inherent emulsification and stabilization properties. Moreover; the emulsification property of gum arabic is further enhanced due to molecular flexibility which allows greater surface interaction with oil droplets.

Gum arabic is also widely used in the food and drink industry to stabilize flavors and essential oils, e.g. in soft drink concentrates. Moreover, gum arabic is used in confectionery such as traditional hard gums, pastilles and as a foam stabilizer in marshmallows. The gum arabic glycoprotein possesses a flexible but compact conformation. It is readily water soluble to give relatively low viscosity Newtonian solutions even at high concentrations (20-30 wt %).

Gums closely related to gum arabic include gum tragacanth, gum karaya and mesquite gum. Gum tragacanth (Astragalus gummifer) is a related exudate gum consisting of a mixture of polysaccharides including an arabinogalactan containing a-L-arabinofuranose and 1-4-linked b-D-galatactopyranose and an acidic complex poly-1-4-linked a-D-galalacturonate. Gum tragacanth is used as an acid-resistant thickener and emulsifier in sauces, salad dressings and confectionery lozenges. Gum karaya (Sterculia urens) has similar physical properties but consists of a-D-galacturonic acid/a-L-rhamnose backbone with b-D-galactose and b-D glucuronic acid side chains. Mesquite gum (Prosopis) consists essentially of an arabinogalactan with a b-1,3-galactopyran core and L-arabinose side chains.

Preferably, the disclosed compositions are aqueous compositions containing gum arabic. In use, the composition is distributed by a suitable mechanism into the air or onto the surface where the malodors exist. The composition is preferably a solution but may also be an emulsion or even a suspension. It is to be understood that the physical presence of the composition should not be considered as limiting the scope of this disclosure as long as such physical presence is compatible with the distributing and odor counteracting properties of the composition.

Without being bound by any particular theory, it is contemplated that gum arabic may remove the malodor ant(s) by a “caging” mechanism in a way similar to that of cyclodextrins. More specifically, gum arabic has a spiral structure and malodorous molecules are trapped in the spiral structure, while cyclodextrins have a toroid shape and malodorous molecules are trapped in the cavities of the toroid structure.

Moreover, as gum arabic also contains chemically active proteins and/or enzymes, it may also remove the malodorant(s) by the odor modification mechanisms discussed above. In one embodiment, the disclosed composition removes a malodorant having an allyl alcohol structure by isomerizing the malodorant into a ketone. Other modifications of the malodorant(s) may also be accomplished by gum arabic and are contemplated herein.

In a preferred embodiment the gum arabic is present in the disclosed composition at a concentration of from about 0.1 to about 50 wt %, more preferably from about 1 to about 10 wt %, and most preferably from about 1 to about 5 wt %. Concentrations of gum arabic higher or lower than the range specified above may also be used in the disclosed composition and should be considered within the scope of this disclosure.

To facilitate the solubilization of gum arabic or other ingredients in the disclosed composition, the composition may further comprise a co-solvent such as an alcohol. The content of the alcohol preferably ranges from about 1 to about 20 wt %. In a refinement, the alcohol is a low molecular weight monohydric alcohol and is present in an amount of from about 5 to about 10 wt %.

The preferred alcohol is ethanol because of its low cost and acceptable odor. Other monohydric alcohols, however; may also be used, including, but not limited to, propanol, isopropanol and butanol. In addition to monohydric alcohols, one of more glycols may also be used as the co-solvent. Suitable glycols include, but are not limited to, ethylene glycol, diethylene glycol, triethylene glycol and tetraethylene glycol. Other organic solvents such as acetone may also be used as the co-solvent. It is to be understood that the type and level of the co-solvent suitable for use in the disclosed composition would be apparent to one of ordinary skill in the art and should not be considered as limiting the scope of this disclosure.

Optionally, the disclosed composition further includes one or more surfactants to facilitate the solubilization of gum arabic or other ingredients in the composition and/or to improve the odor removal performance of the composition. The surfactant(s) may be selected from the group consisting of anionic surfactants, cationic surfactants, nonionic surfactants, amphoteric surfactants, and zwitterionic surfactants. A preferred surfactant is sodium dodecyl sulfate. In a preferred embodiment, the disclosed composition comprises about 1 wt % sodium dodecyl sulfate (a.k.a. sodium lauryl sulfate).

Other surfactants, either ionic or nonionic or a mixture of both, can also be used. Suitable nonionic surfactants may include, but not limited to hydrogenated castor oils, polyglycol ethers and other nonionic surfactants apparent to those skilled in the art. The ionic surfactant can be either cationic or anionic. Suitable cationic surfactants may include a wide variety of quaternary ammonium salt and other cationic surfactants commonly known in the art.

While anionic surfactant disclosed herein is sodium dodecyl sulfate, other salts of an acid with a long chain alkyl group as well as other commonly used anionic surfactants apparent to those skilled in the art, can also be used. Amphoteric and zwitterionic surfactants suitable for use in the disclosed composition may also be included. It is to be understood that the inclusion of the surfactant(s) is optional and that one of ordinary skill in the art would be able to determine the type and level of the surfactant(s) suitable for inclusion in the composition.

In an improvement, the disclosed composition further includes an additional malodor counteractant for further improving the odor removal performance of the composition. Preferably, the additional malodor counteractant has a synergic effect with gum arabic depending on the specific malodorant that is being treated. In one embodiment, the additional malodor counteractant comprises an oxidant such as iodine. In a preferred embodiment, iodine is present in the composition at a concentration of from about 0.001 to about 1 wt %.

In a refinement of this concept, gum arabic and iodine are dissolved in a solvent mixture of an alcohol and water, wherein the alcohol improves the solubility of iodine. In a preferred embodiment, the disclosed composition comprises about 3 wt % gum arabic, about 0.02 wt % iodine, about 1 wt % sodium lauryl sulfate, and the balance being water.

Oxidants other than iodine may also be included in the composition to improve the malodor removal performance thereof. Such oxidants include, but are not limited to, hydrogen peroxide, alkyl hydroperoxides such as t-butyl hydroperoxide, di-alkyl peroxides such as di-t-butyl peroxide, percarboxylic acids such as peracetic acid, oxidative metal salt such as silver carbonate, other peracids such as periodic acid or salts thereof, etc. If the inclusion of oxidants, over time, causes destabilization of the composition by slowly oxidizing one or more components, such as alcohols, gum arabic and/or other additives, the oxidants may be added to the solution immediately before its application to minimize this undesired destabilization effect.

In addition to the oxidants, other additional malodor counteractants well known in the art may also be included in the disclosed composition, provided such inclusion would preferably not affect the purported application of the composition. Thus, the level of the additional malodor counteractant included in the composition is preferably non-staining and non-toxic, and is preferably compatible with other ingredients in the composition. It is to be understood that the type and concentration of the additional malodor counteractant suitable for use in the composition would be apparent to those of ordinary skill in the art and should be considered within the scope of this disclosure.

As a further improvement of the disclosed composition, the gum arabic may be modified to improve its odor removal performance. In one embodiment, the disclosed composition may comprise gum arabic modified by replacing hard water cations contained therein, such as calcium and magnesium, with softer cations such as sodium and/or potassium. Such modification can be accomplished by an ion-exchange process, in which a solution of gum arabic is either stirred with an ion-exchange resin or forced through an ion-exchange column. Other ion-exchange process would be apparent to those skilled in the art without undue experimentation. In a preferred embodiment, the disclosed composition comprises modified gum arabic that is substantially free of hard water cations.

In another embodiement, the disclosed composition comprises gum arabic modified by converting pendent hydroxyl groups of the gum arabic to ether-linked epoxides and/or carboxylic acids. The ether-linked epoxides can be form by an ether-formation reaction in which the pendent hydroxyl groups are alkylated with an epoxy-containing group. The epoxide derivatives of gum arabic improve the malodroant removal performance by reacting with a wide range of malodorous molecules and rendering them innocuous.

The carboxylic acid can be formed by oxidation of the pendent hydroxyl groups into carboxyl groups by any conventional oxidation methods. The resulting carboxylic acid derivatives of gum arabic improve the malodorant removal as the carboxylic groups react with a wide range of basic malodorous molecules and tender them innocuous. Other chemical modifications of the pendent hydroxyl groups of gum arabic, such as by converting the pendent hydroxyl group into other functional groups, or complexing or cheating the hydroxyl group with other molecules or ions, are also available.

It is to be understood that the methods or processes for modifying gum arabic in order to tender it more suitable for odor removal would be apparent to those of ordinary skill in the art and should not be considered as limiting the scope of this disclosure.

In use, the disclosed composition is distributed into the air or onto the surface where the malodor exists. The gum arabic then functions to remove or eliminate the malodor upon contact with the malodorant(s). The composition may be distributed by any conventional methods or devices including, but not limited to, trigger sprayers, pump sprayers, aerosol devices, atomizers, sprinklers, liquid applicators, mistifiers, etc. It is to be understood, however, that the method and apparatus suitable for distributing the composition would be apparent to those of ordinary skill in the art and should not be considered as limiting the scope of this disclosure.

To evaluate the odor removal performance of the disclosed composition, exemplary compositions according to this disclosure are prepared and tested against various malodorants and malodors. Parallel tests are also conducted under identical experimental conditions but using known deodorizing products, such as those containing cyclodextrin, instead of the exemplary compositions. Some exemplary compositions used in the evaluation tests are listed below.

| | | |
|---|---|---|
| Wt % | Name/Formula | Function |

Composition A

| | | |
|---|---|---|
| 98.5 | 10% ethanol in water | solvent/carrier |
| 1.5 | gum arabic | malodor counteractant |

Composition B

| | | |
|---|---|---|
| 97.0 | 10% ethanol in water | solvent/carrier |
| 3.0 | gum arabic | malodor counteractant |

Composition C

| | | |
|---|---|---|
| 98.48 | 10% ethanol in water | solvent/carrier |
| 1.5 | gum arabic | malodor counteractant |
| 0.02 | iodine | malodor counteractant |

Composition D

| | | |
|---|---|---|
| 95.98 | 10% ethanol in water | solvent/carrier |
| 3.0 | gum arabic | malodor counteractant |
| 0.02 | iodine | malodor counteractant |
| 1.0 | sodium lauryl sulfate | surfactant |

Methods and equipments used in conducting the evaluation tests are discussed in greater detail below.

Head Space Analysis

Removal of malodorant with dry gum arabic powder and/or an aqueous solution comprising gum arabic (GA) is measured in the head space of a closed 20 mL rubber septum capped vial. In each case, ground level concentration (GLC) response to malodorant concentration in the head space, obtained by GC-MS analysis of a head space sample, is compared to that of a vial containing the malodorant or a solution of the malodorant in the same solvent as used to dissolve gum arabic. Unless otherwise noted, the head space is approximately 12.5% of saturation in malodor.

Preparation of Air Saturated with a Malodorant

A 1 L or 5 L Tedlar Bag with rubber septum (SKC cat #231-1 or 232-5 septum with single pp fitting with eyelets) is filled to approximately 75% of its total volume with air. The septum fitting is removed and liquid malodorant is added through a micro pipette. The rubber septum is then reattached and the bag is allowed to sit for 24 hours.

Reduction of the Malodorant with Dry Gum Arabic

Gum arabic is added to a 20 mL vial in amounts ranging from 0.00 g to 1.00 g. The vial is capped with a rubber septum and 2.5 mL of air is withdrawn from the head space with a Hamilton Gastight Syringe. To this vial is injected 2.5 mL of air saturated with malodorant (removed from the Tedlar bag and through the septum) to provide a head space that is approximately at 12.5% saturation in malodorant. After approximately 18 hours the reduction in head space malodorant is measured by GC-MS.

Reduction of the Malodorant with the Disclosed Composition

1.00 mL of a solution comprising gum arabic is added to a 20 mL vial. The vial is capped with a rubber septum and 2.5 mL of air is withdrawn from the head space with a Hamilton Gastight Syringe. To this vial is injected 2.5 mL of air saturated with malodorant to provide a head space that is approximately at 12.5% saturation in malodorant. After approximately 18 hours the reduction in head space malodorant is measured by GC-MS.

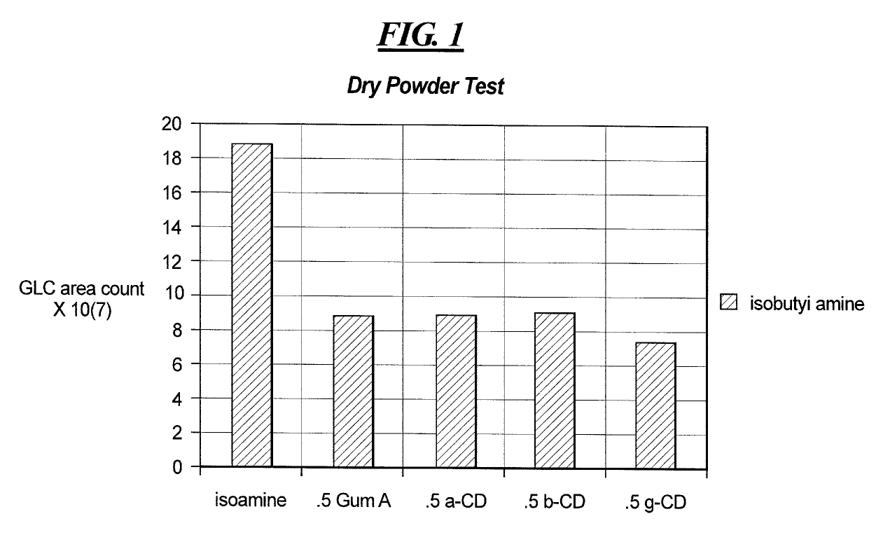

Results of the evaluation tests are illustrated in FIGS. 1-9, and further discussed in greater detail below.

In order to show proof of principal, dry gum arabic power is used in a series of experiments designed to test the efficacy of gum arabic to remove malodors. The use of powder allows the removal of malodorants by gum arabic only thereby excluding any combined effects from solvents and other additives such as surfactants, etc.

A total of six common odoriferous compounds, selected from a broad range of malodorants, are tested against dry gum arabic powder for its odor removing abilities. The six compounds include butylic acid, indene, dimethyl disulfide, pentanethiol, pyridine and iso-butylamine, each representing a chemical class of malodorants commonly presented in a household. In all cases dry gum arabic powder shows removal of malodorant from surrounding air indicated by the head space analysis.

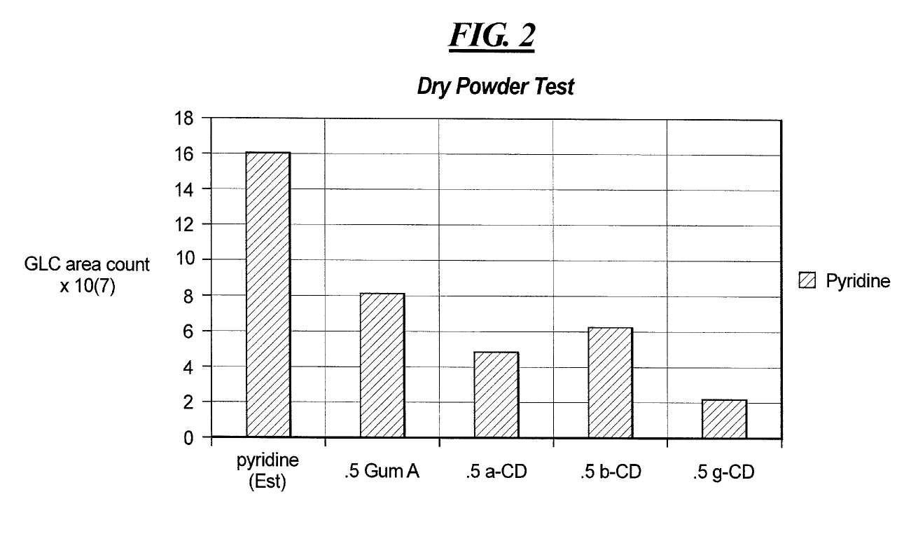

As illustrated in FIG. 1, iso-butylamine is reduced by 53% with 0.5 gram of dry gum arabic, which is approximately on the same level as reductions achieved with 0.5 gram of cyclodextrin (CD). Similar result is achieved when dry gum arabic is tested against pyridine (FIG. 2). Although this dry powder study provides relatively straightforward evaluation of malodor removal performance, a surface area effect of the powder may complicate the quantitative interpretation of the test results. Nevertheless, these dry powder tests clearly demonstrate the ability of gum arabic to remove a wide spectrum of malodorants.

For more accurate evaluation of the malodor removal performance of gum arabic, an aqueous solution of gum arabic is tested against the malodorants discussed above. Unlike the dry gum arabic powder, the application of solubilized gum arabic, either as a solution in water or as a solution in a mixture of an alcohol and water, eliminates any surface area effect. Therefore, any reduction in malodorant can be used to quantitatively evaluate the inherent ability of gum arabic to remove that malodorant.

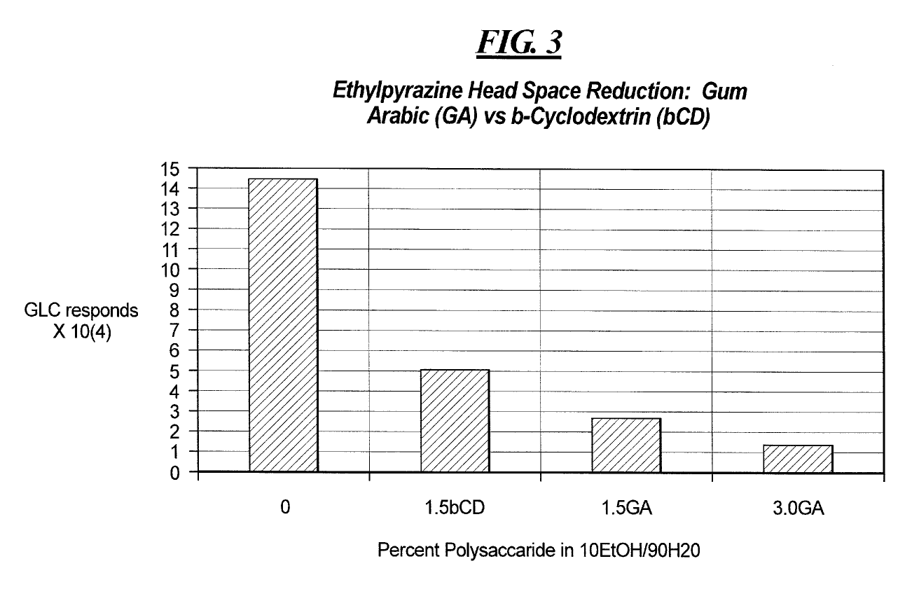

As illustrated in FIG. 3, solutions of gum arabic in a mixture of 10 wt % of ethanol and 90% of water are used to remove ethylpyrazine, a malodorant commonly found in cat urine and wet dog hair. The head space analysis indicates that 82% ethylpyrazine is removed by Composition A, which is greater than that removed by a b-cyclodextrin (b-CD) solution at the same concentration. Moreover, ethylpyrazine removal is improved by increasing the concentration of gum arabic from 1.5 wt % to 3.0 wt % (Composition B).

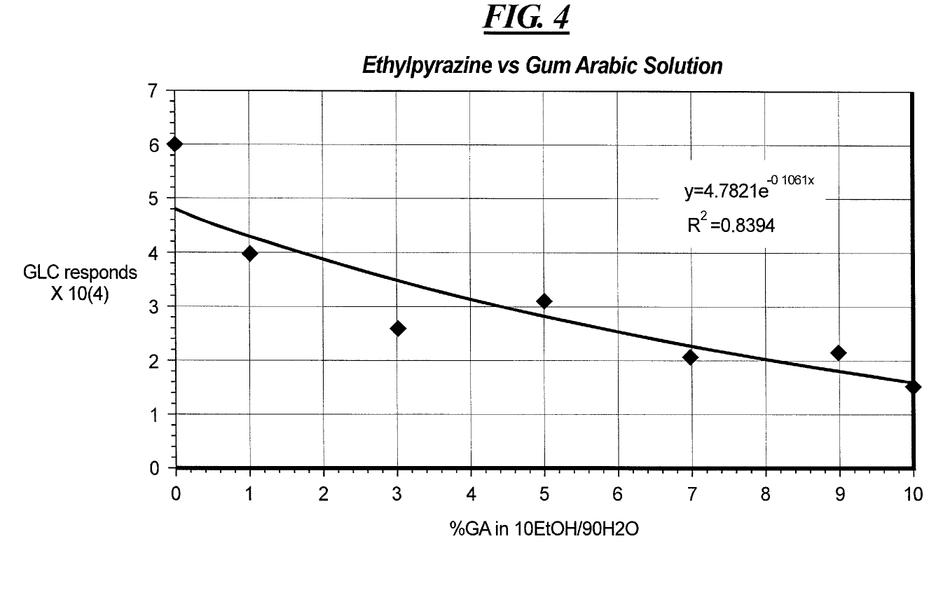

The relationship between the concentration of gum arabic and malodorant removal performance of the composition is further illustrated in FIGS. 4-6, in which solutions of gum arabic in a mixture of 10 wt % ethanol and 90 wt % water are used to remove ethylpyrazine, iso-butylamine and pyridine.

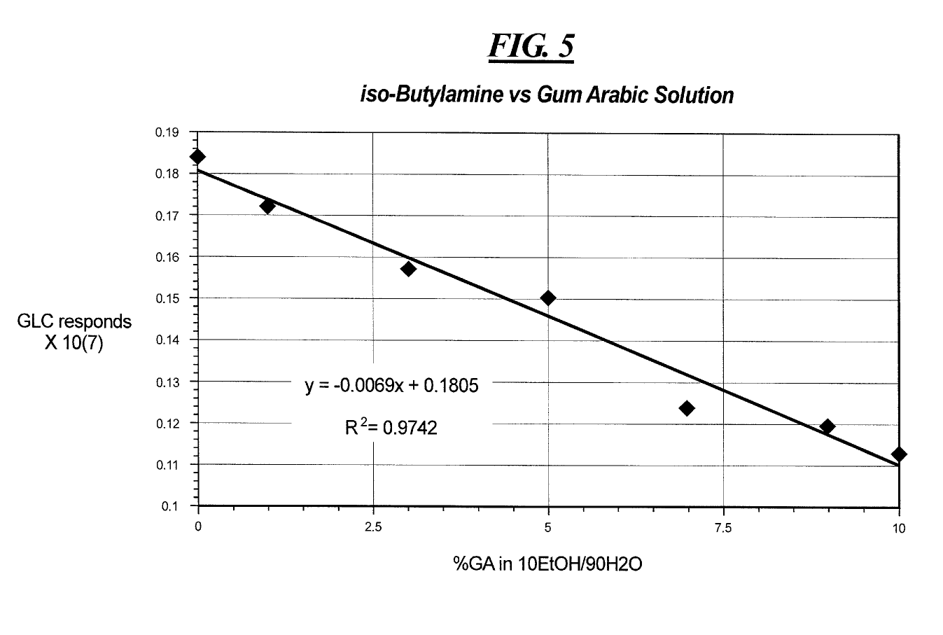

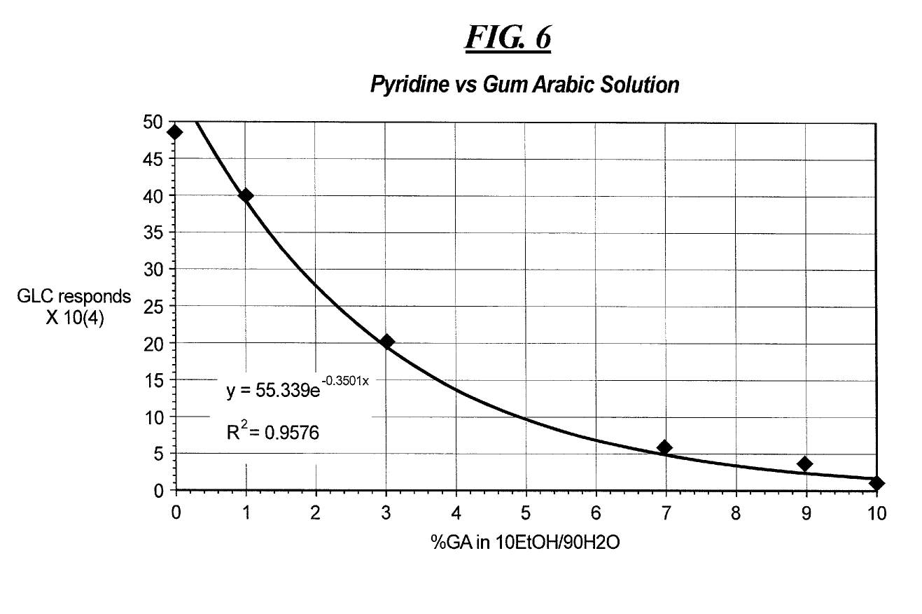

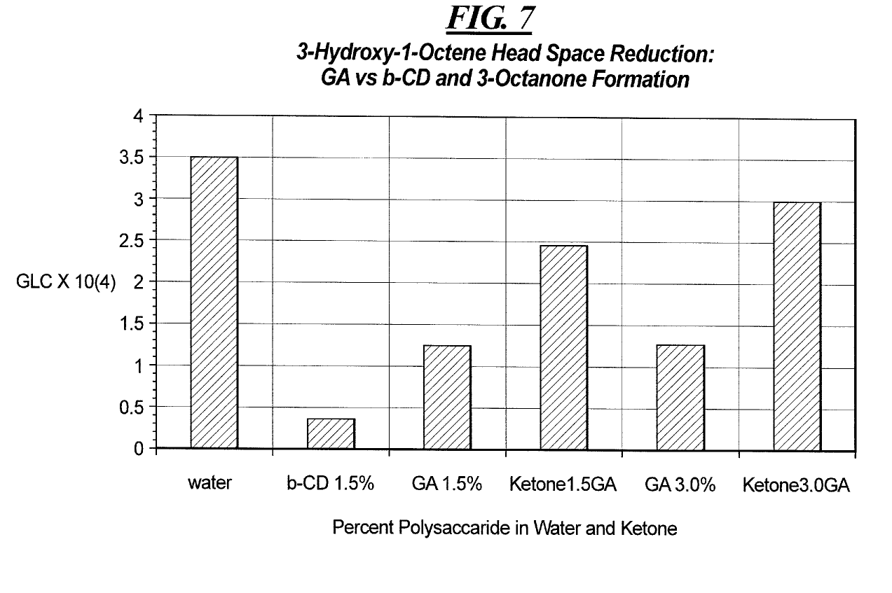

While the disclosed composition may exhibit a linear relationship between the malodorant removal and the concentration of gum arabic when tested against some malodorants such as iso-butylamine (FIG. 5), the relationship may become near-linear or even exponential when the same composition is tested against other malodorants, as in the case of ethylpyrazine and pyridine shown in the examples of FIGS. 4 and 6, respectively. Nevertheless, FIGS. 4 and 6 clearly indicate that malodorant removal is improved with increased concentration of gum arabic. A gum arabic content of 3 wt % is preferred due to a compromise between the malodor ant removal performance and cost efficiency thereof.

As discussed above, gum arabic may also remove the malodorant(s) by one or more odor modification mechanisms. As illustrated in FIG. 7, solutions of gum arabic in water or ketone are used to remove 3-hydroxy-1-octene, a malodorant found in common household molds. An observation arises when gum arabic converts a substantial proportion of 3-hydroxy-1-octene to 3-octanone, possibly through a double bond migration-tautomerization mechanism. It should be noted that other malodorants subject to similar chemical transformation or other chemical transformations may also be effectively removed by the disclosed composition through the one or more odor modification mechanisms.

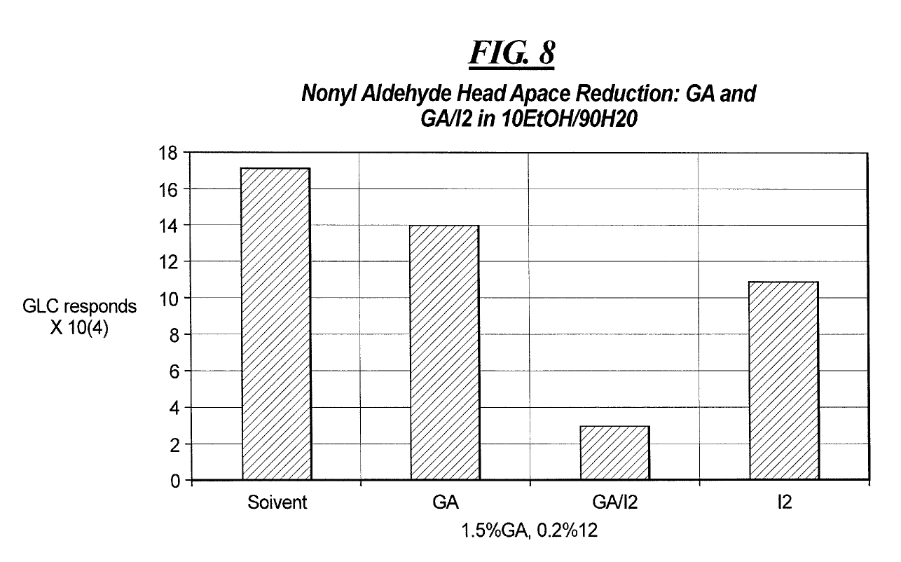

Additional malodor counteractnts may further improve the malodor removal performance of the disclosed composition. In an embodiment an oxidant, such as iodine, is included in the composition to improve the removal of certain malodorant(s) that does not exhibit a significant response to gum arabic alone. As illustrated in FIG. 8, nonyl aldehyde exhibits a relatively low 18% head space removal with 1.5 wt % gum arabic solution in a mixture of 10 wt % ethanol and 90 wt % water. On the other hand, a solution of 0.02 wt % iodine in the same solvent system but without the presence of gum arabic removes 35% of the aldehyde.

When a combined 1.5 wt % gum arabic and 0.02 wt % iodine solution in the same solvent system (Composition C) is used, however, 82% of the aldehyde is removed (FIG. 8). While the exact mechanism of the synergetic effect remains uncertain, one possible explanation is that the effect is a result of the generation of a hypoiodite species by reaction between iodine and hydroxyl groups on gum arabic.

The inclusion of iodine is particularly important when the composition needs to remove an oxidizable malodorant that is not very responsive to gum arabic. In such cases, iodine included in the composition may remove the maldorant be oxidizing the malodorant and rendering it innocuous.

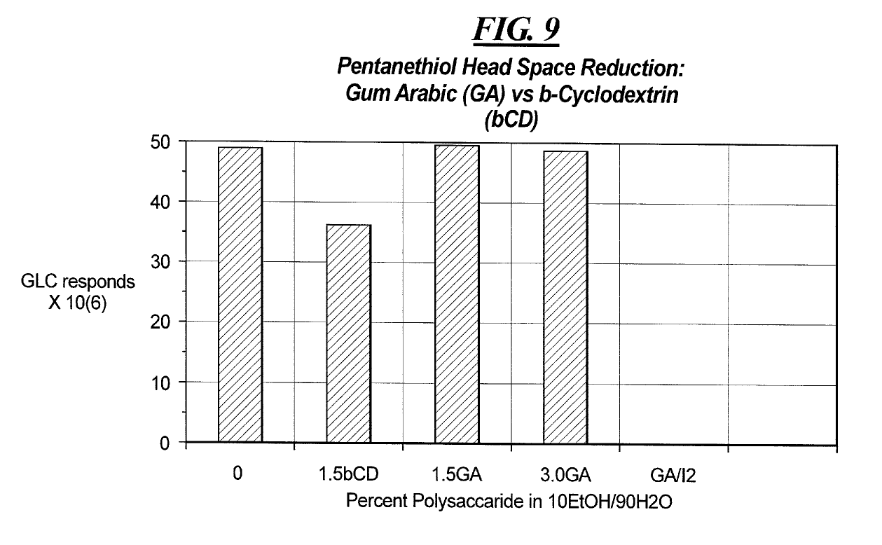

As illustrated in FIG. 9, pentanethiol is difficult to be removed by a deodorizing composition containing 1.5 or 3 wt % gum arabic but without iodine. Moderate malodrant removal is achieved by a comparison composition containing 1.5 wt % cyclodextrin. With the addition of iodine, however, all pentanethiols are almost completely removed from the head space by the gum arabic composition. Without being bound by any particular theory, it is contemplated that pentanethiol may be oxidized by iodine to form a disulfide, which is less odoriferous than the thiol. Other chemical reactions initiated and/or facilitated by iodine may also take place during the removal of the malodorant(s) and should be considered as within the scope of this disclosure.

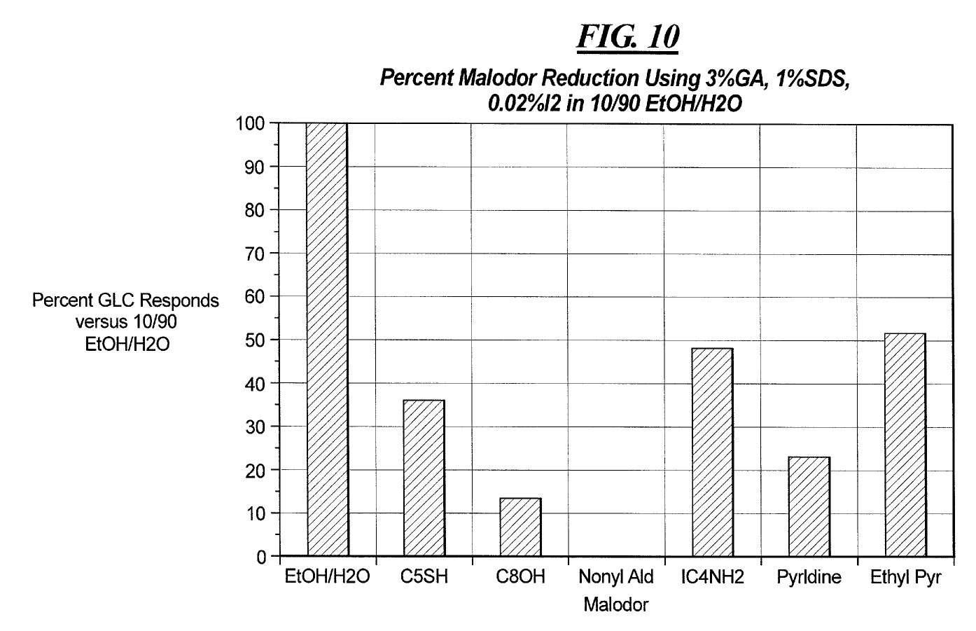

To further improve the malodor removal performance of the disclosed composition, one or more surfactant may be optionally included in the composition. As illustrated in FIG. 10, a solution of 3 wt % gum arabic, 1 wt % sodium lauiyl sulfate, 0.02 wt % iodine in 10% ethanol (Composition D) is used to remove a wide spectrum of malodor ants, which includes pentanethiol (C₅SH), 3-hydroxy-1-octene (C₈OH), nonyl aldehyde, iso-butylamine (iC₄NH₂), pyridine and ethyl pyrazine. In all cases, reduction of more than about 50% of the malodorants is achieved by Composition D (FIG. 10).

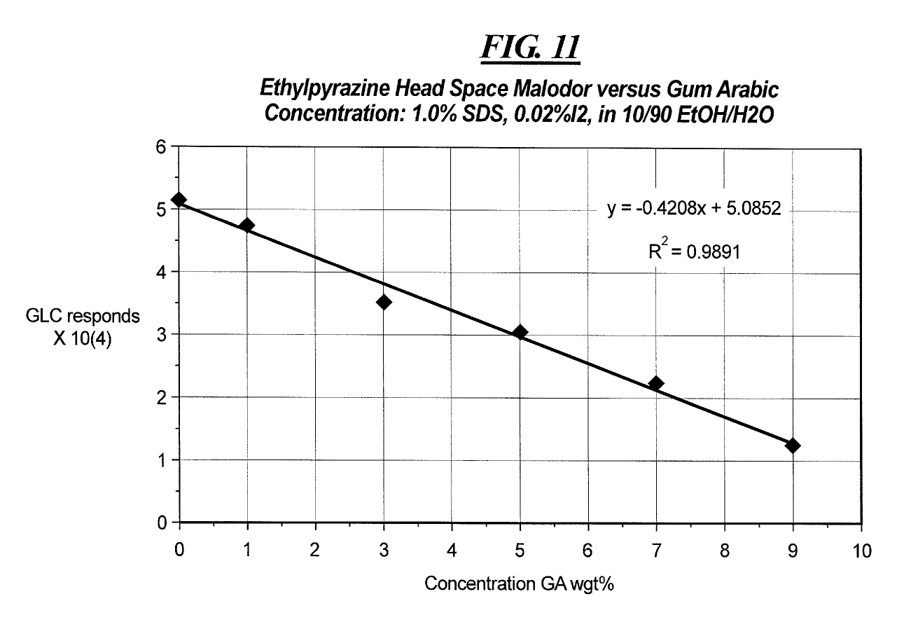

An overall enhancement of iodine and the surfactant is demonstrated in FIG. 11, especially when compared to FIG. 4, which illustrate the removal of the same malodorant (ethylpyrazine) but without the enhancement of iodine or the surfactant. As illustrated in FIGS. 4 and 11, the relationship between the concentration of gum arabic in the composition and the malodorant removal performance thereof is more linear with the addition of iodine and the surfactant. Moreover the odor removal performance of the composition is enhanced with the inclusion of iodine and the surfactant, especially when the composition contains higher amounts of gum arabic.

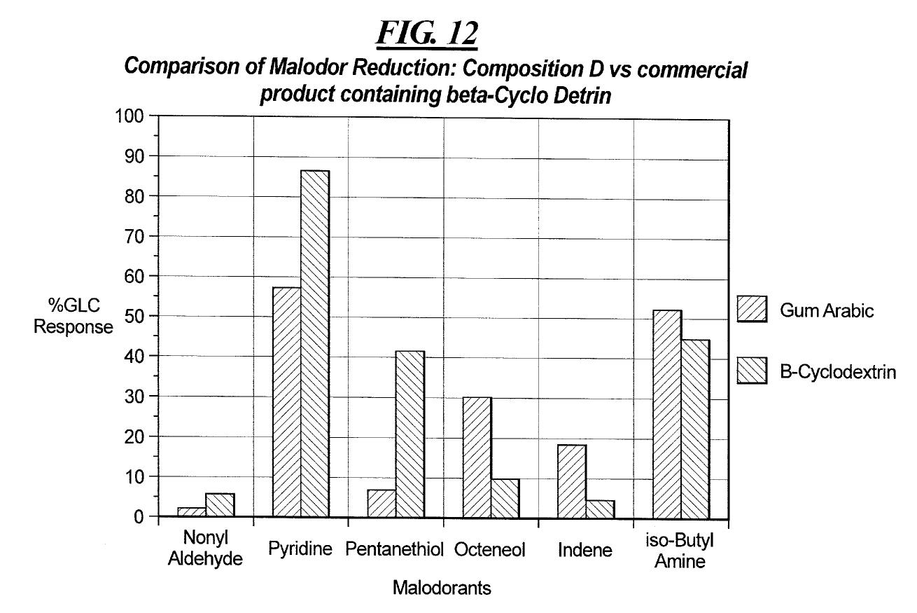

When compared to a commercial deodorizing composition that uses beta-cyclodextrin as the malodor counteractant, as illustrated in FIG. 12, Composition D exhibits better malodrant removal performance against nonyl aldehyde, pyridine, and penanethiol, as well as comparable performance against iso-butyl amine. When tested against octeneol and indene, however, the malodorant removal performance of Composition D is less satisfactory than the commercial composition. It is to be understood, of course, that Composition D may be further optimized to improved the malodorant performance thereof. Such optimization could be accomplished by one of ordinary skill in the art according to this disclosure without undue experimentation.

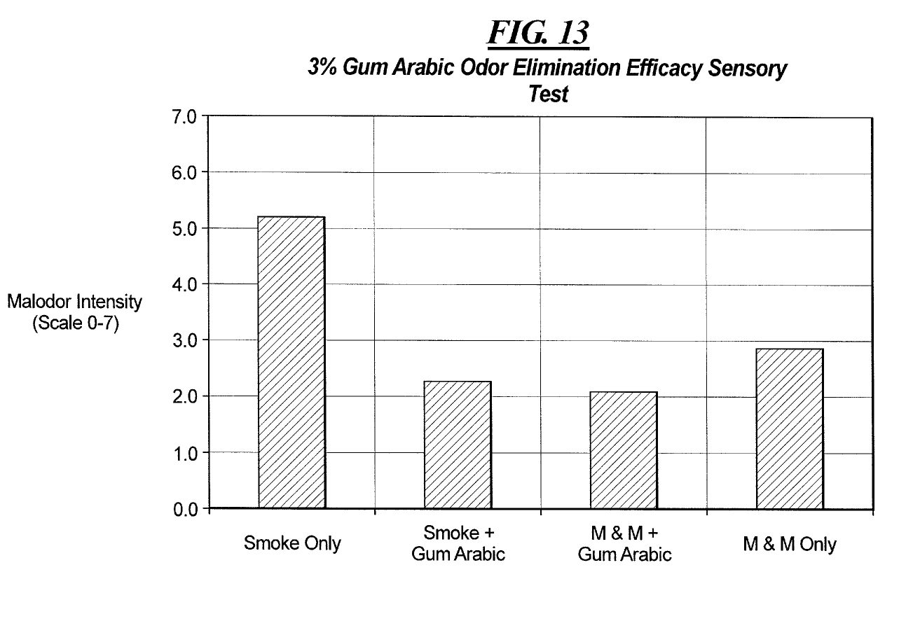

To evaluate the overall performance of the disclosed composition on common malodors, a sensory test is designed to evaluate the effectiveness of the composition to remove smoke malodor and mold/mildew malodor, respectively. In the test, smoke malodor, smoke malodor with 0.88 gram of Composition D, mold and mildew malodor, and mold and mildew malodor with 0.87 gram of Composition D is placed in four jars, respectively. The jars are allowed to equilibrate for one hour prior to sensory test, in which the malodor intensity is evaluated on a 0-7 scale by 15 subjects. The average of the malodor intensity is presented in FIG. 13. Smoke malodor is substantially removed by the aforementioned solution as the malodor intensity is reduced by more than 50%. Mold and mildew malodor is also reduced, although not as significant as the smoke malodor.

In order to compare the malodor removal performance of the disclosed composition, five sets of sensory tests are conducted against five malodors: smoke malodor, low mold and mildew odor (25 μL), high mold and mildew odor (500 μL), low cat urine odor (50 μL), and high cat urine odor (500 μL). In each set of sensory test, perception of the respective malodor on upholstery is evaluated by a panel of professional evaluators before and after the application of five different deodorizing compositions on the upholstery. For reference purposes, a control evaluation using non-odorous upholstery is also included in each set of the sensory test.

The five deodorizing compositions used in each set of the sensory test P1-P5 listed below.

P1 is a fragrance-free deodorizing composition using 1H₂O₂ as the malodor counteractant;

P2 is an aqueous solution (0.1 M) of NaHCO₃;

P3 is de-ionized water;

P4 is Composition D listed above; and

P5 is a fragrance-free deodorizing composition using triethylene glycol (TEG) as the malodor counteractant.

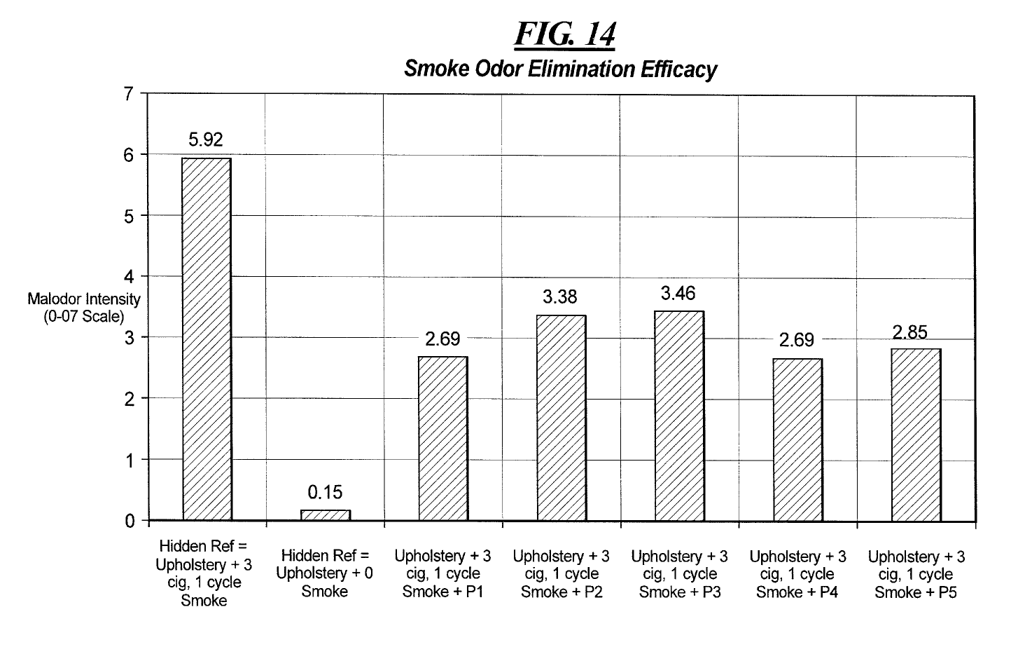

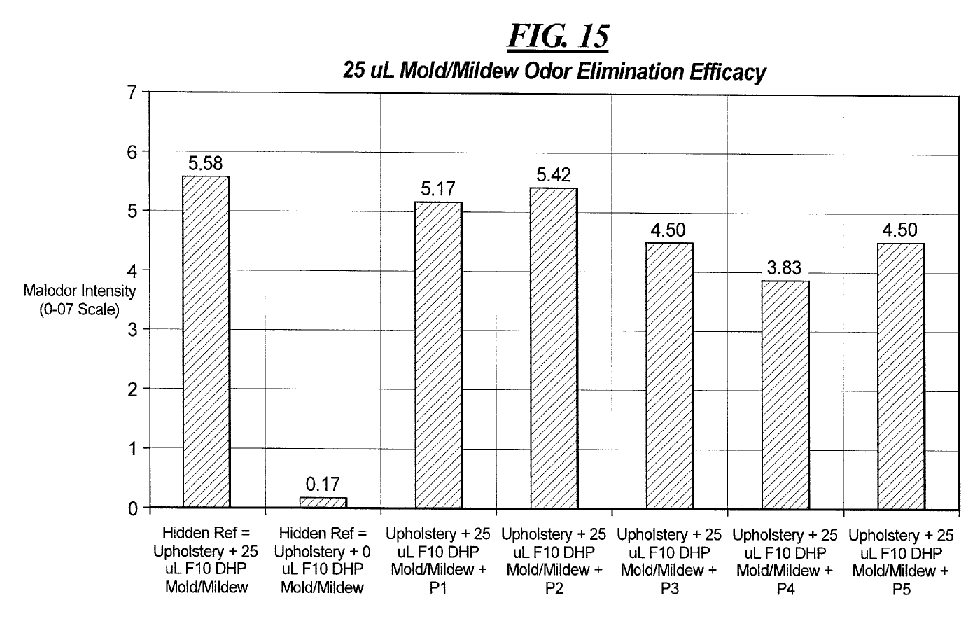

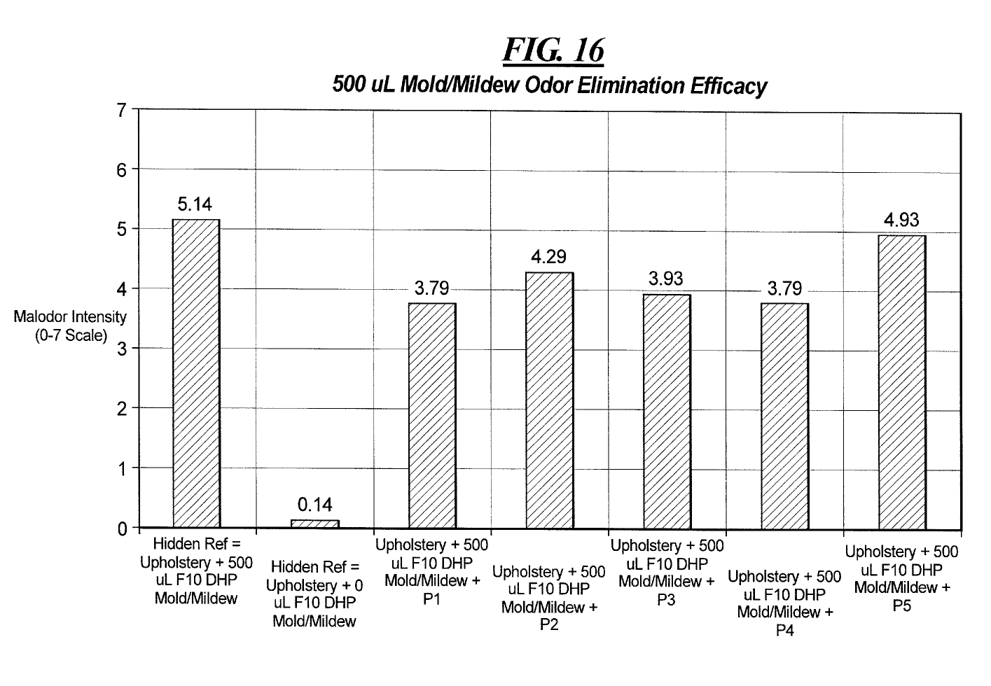

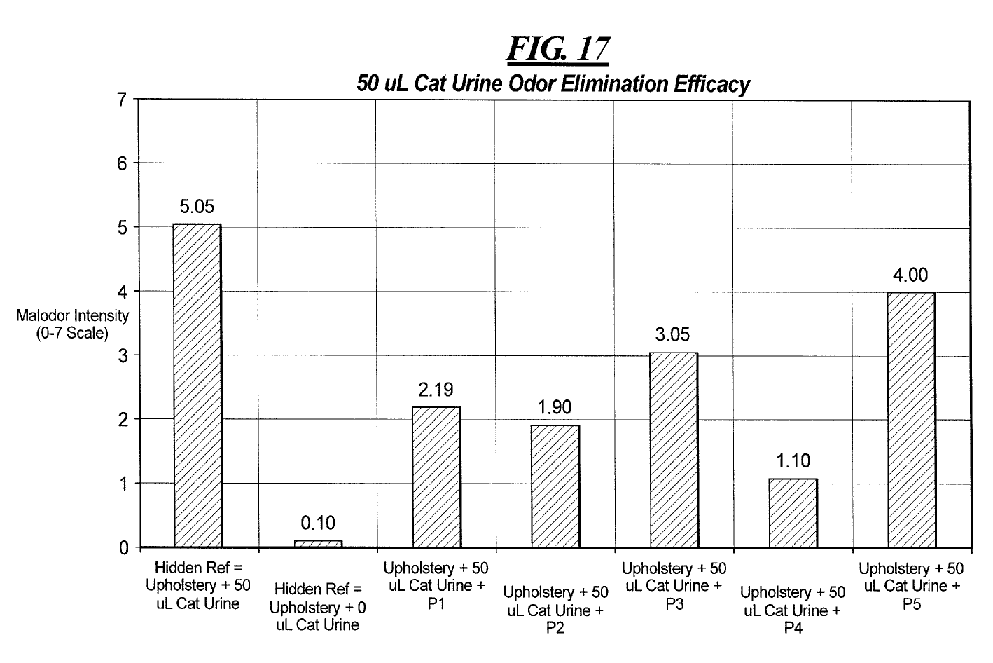

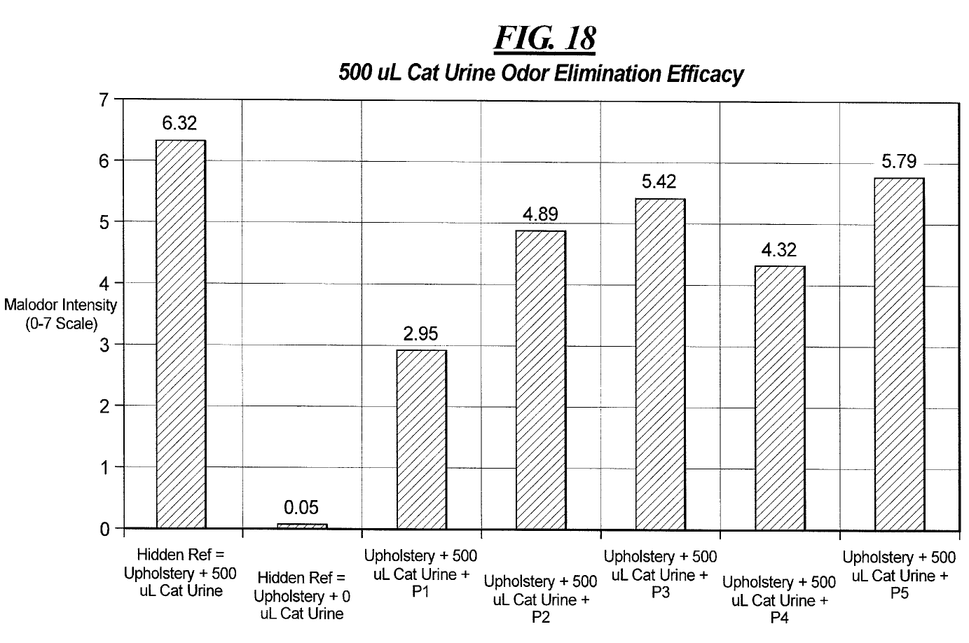

The results of the comparative sensory tests are illustrated in FIGS. 14-18, which clearly demonstrate that the odor removal performance of P4 is better than P2, P3, and P5 when tests against each of the five malodors. When compared to P1, however, P4 exhibits better odor removal performance when tested against low mold and mildew odor, low cat urine odor, as well as similar performance when tested against smoke odor and high mold and mildew odor. When tested against high urine odor, however; P4 is less effective than P1. Nevertheless, the above sensory testes clearly indicate that the disclosed composition is generally more effective in removing various malodors than existing deodorizing compositions that do not use gum arabic as a primary malodor counteractant.

While only certain embodiments have been set forth, alternative embodiments and various modifications will be apparent from the above descriptions of those skilled in the art. These and other alternatives are considered equivalents and within the spirit and scope of this disclosure.

What is claimed:

1. A deodorizing composition comprising:
gum arabic as a malador counteractant in an amount ranging from about 0.1 to about 50 wt %; and
a solvent comprising water.

2. The composition of claim 1 wherein the solvent is a mixture of water and an alcohol.

3. The composition of claim 2 wherein the alcohol is ethanol.

4. The composition of claim 2 wherein the mixture comprises from about 90 to about 95 wt % water and about 5 to about 10 wt % alcohol.

5. The composition of claim 1 wherein the solution is substantially free of hard water cations.

6. The composition of claim 1 wherein the pendent hydroxyl groups of the gum arabic are at least partially converted to ether-linked epoxide groups.

7. The composition of claim 1 wherein hydroxyl groups of the gum arabic are at least partially converted to carboxylic acid groups.

8. The composition of claim 1 wherein the composition further comprises iodine.

9. The composition of claim 8 wherein the iodine is presented in an amount of from about 0.01 to about 1 wt %.

10. The composition of claim 1 wherein the composition further comprises a surfactant.

11. The composition of claim 11 wherein the surfactant is sodium dodecyl sulfate.

12. A deodorizing composition comprising:
gum arabic in an amount ranging from about 0.1 to about 50 wt %;
a solvent comprising water; and
iodine.

13. The composition of claim 12 wherein the solvent is a mixture of water and an alcohol.

14. The composition of claim 13 wherein the alcohol is ethanol.

15. The composition of claim 13 wherein the mixture comprises from about 90 to about 95 wt % water and from about 5 to about 10% alcohol.

16. The composition of claim 12 wherein the solution is substantially free of hard water cations.

17. The composition of claim 12 wherein the pendent hydroxyl groups of the gum arabic are at least partially converted to ether-linked epoxide groups.

18. The composition of claim 12 wherein hydroxyl groups of the gum arabic are at least partially converted to carboxylic acid groups.

19. The composition of claim 12 wherein the iodine is presented in an amount of from about 0.01 to about 1 wt %.

20. A deodorizing composition comprising:
gum arabic in an amount ranging from about 0.1 to about 50 wt %;
a solvent comprising water;
iodine; and
a surfactant.
